(19) United States
(12) Patent Application Publication
(10) Pub. No.: US 2014/0283849 A1
(43) Pub. Date: Sep. 25, 2014

(54) Jaw Range Limiter and Method for Weight Loss

(71) Applicant: Roger S. Pecina, Niles, MI (US)

(72) Inventors: Roger S. Pecina, Niles, MI (US); Stephen F. Paige, Ocala, FL (US)

(21) Appl. No.: 13/849,177

(22) Filed: Mar. 22, 2013

(51) Int. Cl. A61F 5/058; A61C 7/36

(52) U.S. Cl. CPC A61F 5/05891; A61C 7/36
USPC 128/869

(57) ABSTRACT

A dental apparatus for limiting how far a person can open their mouth to hamper eating is disclosed. A first tooth interface is affixed to at least one upper tooth and a second tooth interface is affixed to at least one lower tooth in proximity to the first tooth interface. A range limiting device having at least two sections, such as two triangular shaped sections, is sequentially linked from a first section of the range limiting device to a last section of the range limiting device. Each section of the range limiting device is movably affixed to an opposing tooth interface, such that the device movably interconnects the first tooth interface and the second tooth interface, thereby allowing the person to completely close their mouth, and limiting the person to opening their mouth to a range less than fully opened.

The invention relates generally to the field of health, and more specifically to an apparatus and system for limiting range between the jaws to hamper eating.

### BACKGROUND

There are known health benefits in moderating caloric intake and not habitually “over-eating”. The benefits include reducing the risk of obesity and its co-morbidity rates for heart disease, diabetes, high blood pressure, cancer, etc. Weight management methods, such as regular exercise, consumption of high energy foods low in calories, and hydration, etc. are all well known. In addition, taking more time to eat is a well known method for improving digestion and reducing the risk of “over-eating”.

According to data from the Centers for Disease Control and Prevention, in the last 30 years, obesity rates of Americans have more than doubled in adults, and tripled in children. For many reasons, genetic and/or psychological susceptibility, coupled with environmental factors, Americans have been super-sized. Notwithstanding all the well-known methods to personally manage one's weight, like not eating late at night, drinking less beer, or cutting out the chocolate cake, etc., human psychology interferes with personal behavior, and inhibits the transformation of these effective methods, into sustainable weight management habits. In response to demand, the market has matched this super-size problem with a buffet of weight loss products and procedures.

Pharmaceutical firms sell over-the-counter (“OTC”) and prescription drugs to make you feel full, increase metabolism, etc. These products have risks and in rare cases have caused death, making such weight loss methods impractical and/or too risky for some. Alternatively, health care providers offer surgically delivered procedures such as lap bands, gastric bypass, sleeve gastrectomy, etc., which aim to reduce stomach capacity. Although such surgeries generally are successful, the inherent risk includes death. Furthermore, the expense of such operations make such methods cost prohibitive for some people.

An attempt has been made at moderating caloric intake using a dental apparatus which is placed in the mouth and worn while eating. It essentially takes up space in the mouth while you chew, drink, and/or swallow food, thereby increasing eating times and frustration. Like an orthodontic retainer which is sometimes only worn at night, this weight loss apparatus is only worn while eating, making it impractical for the following reasons: it can be removed by the wearer making it too easy to stop using, since meals are not always planned it must be carried around making it easy to lose, and impaired communication during meals may require removing it between bites which is neither hygienic, nor convenient, etc.

There are existing ways to fully immobilize the jaws. For example, the mouth can be essentially wired closed. Such a method is intended to minimize aggravation to the mouth and facial region after trauma and stimulate healing. Although in some cases, a small space behind the back teeth can receive a straw to drink, fully immobilizing the jaws to hamper eating seems inhumane even if by choice, and not practical for the following reasons: communication is severely impaired, appearance is inappropriate for some working and/or social environments, and the wearer can not open their mouth at all which inhibits the wearer from receiving adequate nutrition, thereby requiring intravenous nutrition delivery.

Another example of a device which immobilizes the jaws is a helmet-like apparatus worn on the head, and secured around the chin. Essentially like a straight-jacket for the mouth, the wearer donning the “facial straight jacket” can not open their mouth. This device is not practical for weight loss for similar reasons: communication, appearance, comfort, and removability, etc.

What is needed is a semi-permanent dental apparatus not easily removable, that limits the range a wearer can open their mouth, thereby hampering eating.

### SUMMARY

A dental apparatus and system for limiting range between the jaws to hamper eating is disclosed. Range limiting is done by installing in a wearer's mouth at least one range limiting device to limit how far the wearer can open their mouth.

In one embodiment, a dental apparatus for range limiting is disclosed comprising a first tooth interface, an inside surface of the first tooth interface affixed to at least one upper tooth, a second tooth interface, an inside surface of the second tooth interface affixed to at least one lower tooth, and a range limiting device having at least two sections. Each section of the at least two sections of the range limiting device is sequentially linked to another of the sections, from a first section of the range limiting device, to a last section of the range limiting device. The first section of the range limiting device is movably affixed to an outside surface of the first tooth interface and a last section of the range limiting device is movably affixed to an outside surface of the second tooth interface, thereby movably interconnecting the first tooth interface and the second tooth interface to one another, such that the range limiting device permits a wearer to completely close their mouth and the range limiting device limits the wearer to opening their mouth to a range less than fully opened.

In another embodiment, a dental apparatus for range limiting further comprises a first bracket affixed to or integral to the outside surface of the first tooth interface, a first enclosed channel affixed to or integral to the first bracket, a second bracket affixed to or integral to the outside surface of the second tooth interface, a second enclosed channel, and the second enclosed channel affixed to or integral to the second bracket. The first section of the at least two sections of the range limiting device is movably affixed to the first enclosed channel, and the last section of the at least two sections of the range limiting device is movably affixed to the second enclosed channel, thereby movably interconnecting the first tooth interface and the second tooth interface to one another, such that the range limiting device permits the wearer to completely close their mouth and the range limiting device limits the wearer to opening their mouth to a range less than fully opened.

In an alternative embodiment, a dental apparatus for range limiting further comprises a first loop affixed to or integral to the outside surface of the first tooth interface, and a second loop affixed to or integral to the outside surface of the second tooth interface. The first section of the at least two sections of the range limiting device is movably affixed to the first loop, and the last section of the at least two sections of the range limiting device is movably affixed to the second loop, thereby movably interconnecting the first tooth interface and the second tooth interface to one another, such that the range limiting device permits the wearer to completely close their mouth and the range limiting device limits the wearer to opening their mouth to a range less than fully opened.

In an alternative embodiment, a dental apparatus for range limiting further comprises a first C-shaped groove affixed to or integral to the outside surface of the first tooth interface, and a second C-shaped groove affixed to or integral to the outside surface of the second tooth interface, the first section of the at least two sections of the range limiting device is movably affixed to the first C-shaped groove, and the last section of the at least two sections of the range limiting device is movably affixed to the second C-shaped groove, thereby movably interconnecting the first tooth interface and the second tooth interface to one another, such that the range limiting device permits the wearer to completely close their mouth and the range limiting device limits the wearer to opening their mouth to a range less than fully opened.

In an alternative embodiment, a dental apparatus for range limiting comprises a range limiting device having exactly two sections, each of the two sections of the range limiting device is selected from the group consisting of, a triangular shaped section, a semi-circular shaped section, a D-shaped section, a circular shaped section, or an oval shaped section. Each of the two sections of the range limiting device is sequentially linked from the first section of the range limiting device to the last section of the range limiting device, thereby movably interconnecting the first tooth interface and the second tooth interface to one another, such that the range limiting device permits a wearer to completely close their mouth and the range limiting device limits the wearer to opening their mouth to a range less than fully opened.

For example, a first triangular shaped section of a range limiting device is sequentially linked to a last triangular shaped section of the range limiting device. The first triangular shaped section of the range limiting device is movably affixed to a first enclosed channel affixed to or integral to an outside surface of a first tooth interface and the last triangular shaped section of the range limiting device is movably affixed to a second enclosed channel affixed to or integral to an outside surface of a second tooth interface, thereby movably interconnecting the first tooth interface and the second tooth interface, such that the range limiting device permits a wearer to completely close their mouth and the range limiting device limits the wearer to opening their mouth to a range less than fully opened.

In an alternative embodiment, a dental apparatus for range limiting comprises a range limiting device having exactly three sections, where each of a first section and a last section of the range limiting device is selected from the group consisting of, a triangular shaped section, a semi-circular shaped section, a D-shaped section, a circular shaped section, or an oval shaped section. A third section of the range limiting device comprises at least one sub-section, selected from the group consisting of, a circular shaped section, or a semi-circular shaped section.

For example, each of a first triangular shaped section of the range limiting device, a last triangular shaped section of the range limiting device, and three semi-circular shaped sub-sections movably linked to one another, is sequentially linked from the first triangular shaped section of the range limiting device to the last triangular shaped section of the range limiting device. The first triangular shaped section of the range limiting device is movably affixed to a first enclosed channel affixed to or integral to an outside surface of a first tooth interface and the last triangular shaped section of the range limiting device is movably affixed to a second enclosed channel affixed to or integral to an outside surface of a second tooth interface, thereby movably interconnecting the first tooth interface and the second tooth interface, such that the range limiting device permits a wearer to completely close their mouth and the range limiting device limits the wearer to opening their mouth to a range less than fully opened.

In alternative embodiment, a system for range limiting is disclosed comprising a range limiting device having an elongated bar with two protrusive ends. Although not easily removable, at least one of the protrusive ends is removable from either the first end of the elongated bar or the second end of the elongated bar. For example, a protrusive bulb on a first end of an elongated bar is removed and the first end of the elongated bar is sequentially inserted through a first loop affixed to or integral to an outside surface of a first tooth interface. The first end of the elongated bar is then inserted through a second loop affixed to or integral to an outside surface of the second tooth interface. The protrusive bulb from the first end of the elongated bar is re-affixed to the first end of the elongated bar, thereby movably interconnecting the first tooth interface and the second tooth interface to one another, such that the system for range limiting permits complete closure of the mouth of the wearer and the system for range limiting limits the opening of the mouth of the wearer to a range less than fully opened.

In an alternative embodiment, a method for weight loss is disclosed. The method comprises providing a range limiting device having at least two sections. The method includes the step of affixing an inside surface of a first tooth interface to one upper tooth and the step of affixing an inside surface of a second tooth interface to one lower tooth. The method includes the step of sequentially linking each of the at least two sections of the range limiting device from a first section of the range limiting device to a last section of the range limiting device. Before or after the step of sequentially linking each of the at least two sections of the range limiting device, the method includes the step of movably affixing the first section of the at least two sections of the range limiting device to an outside surface of the first tooth interface, and the step of movably affixing the last section of the at least two sections of the range limiting device to an outside surface of the second tooth interface, thereby movably interconnecting the first tooth interface and the second tooth interface to one another, such that the range limiting device permits a wearer to completely close their mouth and the range limiting device limits the wearer to opening their mouth to a range less than fully opened.

The method for weight loss further comprises the first tooth interface having a first enclosed channel affixed to or integral to the outside surface of the first tooth interface and the second tooth interface having a second enclosed channel affixed to or integral to the outside surface of the second tooth interface. The first section of the at least two sections of the range limiting device is movably affixed to the first enclosed channel affixed to or integral to the outside surface of the first tooth interface and the last section of the at least two sections of the range limiting device is movably affixed to the second enclosed channel affixed to or integral to the outside surface of the second tooth interface, thereby movably interconnecting the first tooth interface and the second tooth interface to one another, such that the range limiting device allows a wearer to completely close their mouth and the range limiting device limits the wearer to opening their mouth to a range less than fully opened.

In an alternative embodiment, the method for weight loss further comprises a first tooth interface having a first loop affixed to or integral to the outside surface of the first tooth interface and a second tooth interface having a second loop affixed or integral to the outside surface of the second tooth interface. The first section of the at least two sections of the range limiting device is movably affixed to the first loop affixed to or integral to the outside surface of the first tooth interface and the last section of the at least two sections of the range limiting device is movably affixed to the second loop affixed to or integral to the outside surface of the second tooth interface, thereby movably interconnecting the first tooth interface and the second tooth interface to one another, such that the range limiting device allows a wearer to completely close their mouth and the range limiting device limits the wearer to opening their mouth to a range less than fully opened.

In an alternative embodiment, the method for weight loss further comprises a first tooth interface having a first C-shaped groove affixed to or integral to the outside surface of the first tooth interface and a second tooth interface having a second C-shaped groove affixed or integral to the outside surface of the second tooth interface. The first section of the at least two sections of the range limiting device is movably affixed to the first C-shaped groove affixed to or integral to the outside surface of the first tooth interface and the last section of the at least two sections of the range limiting device is movably affixed to the second C-shaped groove affixed to or integral to the outside surface of the second tooth interface, thereby movably interconnecting the first tooth interface and the second tooth interface to one another, such that the range limiting device allows a wearer to completely close their mouth and the range limiting device limits the wearer to opening their mouth to a range less than fully opened.

In an alternative embodiment, the method for weight loss comprises a range limiting device having exactly two sections and each of the two sections of the range limiting device is selected from the group consisting of, a triangular shaped section, a semi-circular shaped section, a D-shaped section, a circular shaped section, or an oval shaped section. Each of the two sections of the range limiting device is sequentially linked from the first section of the range limiting device to the last section of the range limiting device, thereby movably interconnecting the first tooth interface and the second tooth interface to one another, such that the range limiting device allows a wearer to completely close their mouth and the range limiting device limits the wearer to opening their mouth to a range less than fully opened.

In an alternative embodiment, the method for weight loss comprises a range limiting device having exactly three sections. Each of the first section of the range limiting device and the last section of the range limiting device is selected from the group consisting of, a triangular shaped section, a semi-circular shaped section, a D-shaped section, or a circular shaped section. A third section of the range limiting device is at least one other sub-section selected from the group consisting of, a circular shaped section, or a semi-circular shaped section. Each of the first section of the range limiting device, the last section of the range limiting device, and the third sub-section of the range limiting device is sequentially linked from the first section of the range limiting device to the last section of the range limiting device, thereby movably interconnecting the first tooth interface and the second tooth interface to one another, such that the range limiting device allows a wearer to completely close their mouth and the range limiting device limits the wearer to opening their mouth to a range less than fully opened.

In each of the foregoing embodiments of a method for weight loss, the method further comprises after the step of movably affixing the first section of the range limiting device to the outside surface of the first tooth interface, and the step of movably affixing the last section of the range limiting device to the outside surface of the second tooth interface, the step of periodic monitoring of the wearer and the step of periodic adjusting of the range limiting device, such that the range limiting device allows a wearer to completely close their mouth and the range limiting device limits the wearer to opening their mouth to a range less than fully opened.

In each of the foregoing embodiments of a jaw range limiter and a method for weight loss, the range limiting device is made of materials known in the industry of sufficient strength and gauge, such that after the range limiting device is installed in the mouth of the wearer, the range limiting device is neither easily broken using normal jaw pressure, nor easily removable.

### BRIEF DESCRIPTION OF THE DRAWINGS

The invention can be best understood by those having ordinary skill in the art by reference to the following detailed description when considered in conjunction with the accompanying drawings in which:

FIG. 1A is an exploded view of a first embodiment of the apparatus.

FIG. 1B is an exploded view of a first embodiment of the apparatus showing range limit.

FIG. 2A is a view of a first embodiment of the apparatus installed on one side with the mouth closed.

FIG. 2B is a view of a first embodiment of the apparatus installed on one side with the mouth ajar.

FIG. 3A is a view of a second embodiment of the apparatus installed on one side with the mouth closed.

FIG. 3B is a view of a second embodiment of the apparatus installed on one side with the mouth ajar.

FIG. 4A is a view of a third embodiment of the apparatus installed on one side with the mouth closed.

FIG. 4B is a view of a third embodiment of the apparatus installed on one side with mouth ajar.

FIG. 5A is a view of a fourth embodiment of the apparatus installed on one side with the mouth closed.

FIG. 5B is a view of a fourth embodiment of the apparatus installed on one side with the mouth ajar.

FIG. 6 is an exploded view of a second embodiment of the apparatus.

FIG. 7 is an exploded side view of a third embodiment of the apparatus.

### DETAILED DESCRIPTION

Reference will now be made in detail to the present embodiments of the invention, examples of which are illustrated in the accompanying drawings. Throughout the following detailed description, the same reference numerals refer to the same elements in all figures. In the disclosed embodiments, references made to jaws and arches can be used interchangeably.

Typically people wanting to lose weight attempt to reduce their caloric intake by eating less. However, because eating makes you feel good, and high calorie foods are hard to portion, cheap and easy to find, etc., such methods often fail. And with advertising messages like “Betcha Can't Eat Just One”, too many Americans are biting off more than they should chew. Therefore, by physically hampering the eating process, a person will be less likely to over-eat habitually.

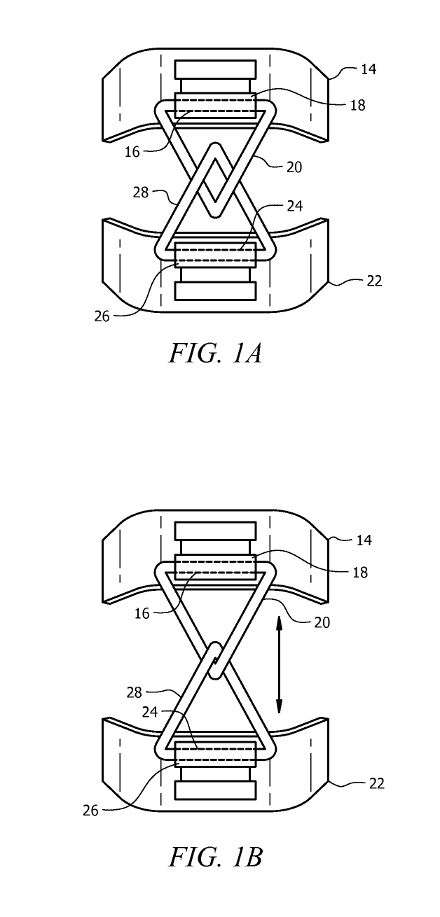

Referring to FIGS. 1A and 1B, a first embodiment of a jaw range limiter will be described. A jaw range limiter is shown having a first tooth interface 14, a first bracket 18 affixed to or integral to the first tooth interface 14, the first bracket 18 having a first enclosed channel 16 affixed to or integral to the first bracket 18, a second tooth interface 22, a second bracket 26 affixed to or integral to the second tooth interface 22, and the second bracket 26 having a second enclosed channel 24 affixed to or integral to the second bracket 26.

A first triangular shaped section 20 of a range limiting device is shown movably linked to a last triangular shaped section 28 of the range limiting device. The first triangular shaped section 20 of the range limiting device is movably affixed to the first enclosed channel 16 and the last triangular shaped section 28 of the range limiting device is movably affixed to the second enclosed channel 24. Preferably, the triangular shaped sections 20, 28 of the range limiting device move freely while linked to one another and while each is movably affixed to both of the first enclosed channel 16 and the second enclosed channel 24. In FIG. 1B, the apparatus shows the limitation of range between the tooth interfaces 14, 22.

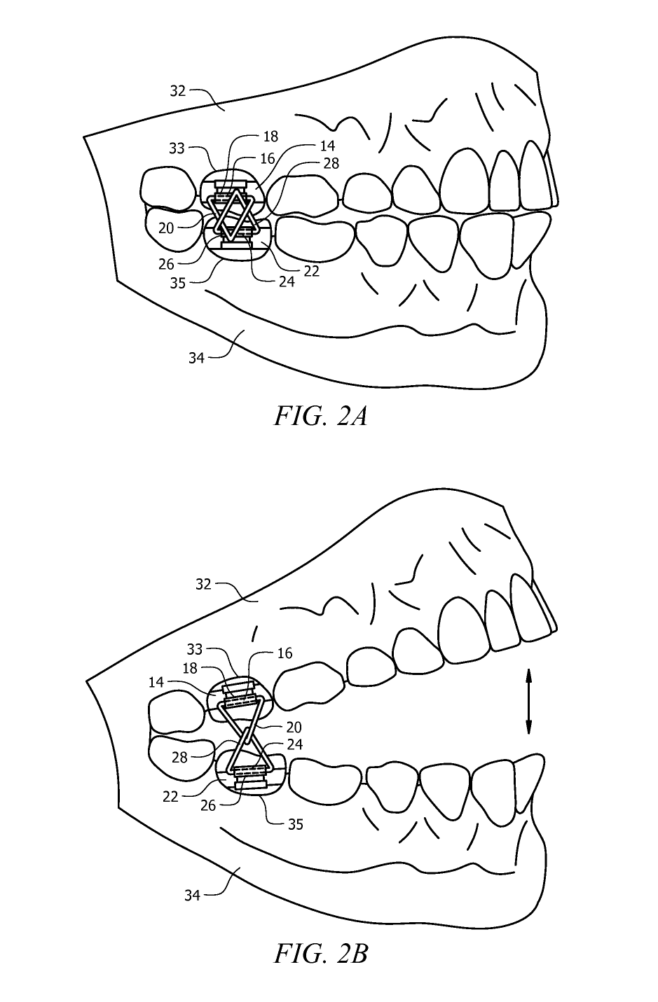

Referring to FIGS. 2A and 2B, a first embodiment of a jaw range limiter installed in a mouth will be described. An inside surface of a first tooth interface 14 is affixed to a first tooth 33 in the upper arch 32, and the inside surface of a second tooth interface 22 is affixed to a second tooth 35 in the lower arch 34. Each tooth interface 14, 22 is affixed in any way known in the industry to at least one tooth and/or faux tooth or dental support apparatus and/or structure, etc., as is known in the industry.

The tooth interfaces 14, 22 are affixed to opposing teeth 33, 35, although other installation locations are anticipated such as a neighboring tooth and/or faux tooth or dental support apparatus and/or structure, etc. as is known in the industry. A first bracket 18 is affixed to or integral to an outside surface of the first tooth interface 14 and a second bracket 26 is affixed to or integral to an outside surface of the second tooth interface 22.

A range limiting device is shown having a first triangular shaped section 20 and a last triangular shaped section 28. The first triangular shaped section 20 of the range limiting device is sequentially linked to the last triangular shaped section 28 of the range limiting device. The first triangular shaped section 20 of the range limiting device is movably affixed to a first enclosed channel 16 affixed to or integral to the first bracket 18. The last triangular shaped section 28 of the range limiting device is movably affixed to a second enclosed channel 24 affixed to or integral to the second bracket 26.

In an alternative embodiment, prior to affixing the inside surface of each tooth interface 14, 22 to at least one tooth, the first triangular shaped section 20 of the range limiting device and the last triangular shaped section 28 of the range limiting device are sequentially linked to one another. Next, the first triangular shaped section 20 of the range limiting device is movably affixed to the first enclosed channel 16 affixed to or integral to the first tooth interface 14, and the last triangular shaped section 28 of the range limiting device is movably affixed to the second enclosed channel 24 affixed to or integral to the second tooth interface 22.

In an alternative embodiment not shown, a first C-shaped groove is affixed to or integral to the first tooth interface 14 and/or a second C-shaped groove is affixed to or integral to the second tooth interface 22. The first triangular shaped section 20 of the range limiting device is movably affixed to the first C-shaped groove and the last triangular shaped section 28 of the range limiting device is movably affixed to the second C-shaped groove affixed to or integral to the second tooth interface 22.

Preferably, the triangular shaped sections 20, 28 of the range limiting device move freely while each is linked to one another and while each is movably affixed to an enclosed channel 16, 24 and/or a C-shaped groove (not shown). In FIG. 2A, the mouth is shown fully closed with the range limiting device installed. In FIG. 2B, the apparatus and system is shown physically limiting the mouth from opening to a range less than fully opened.

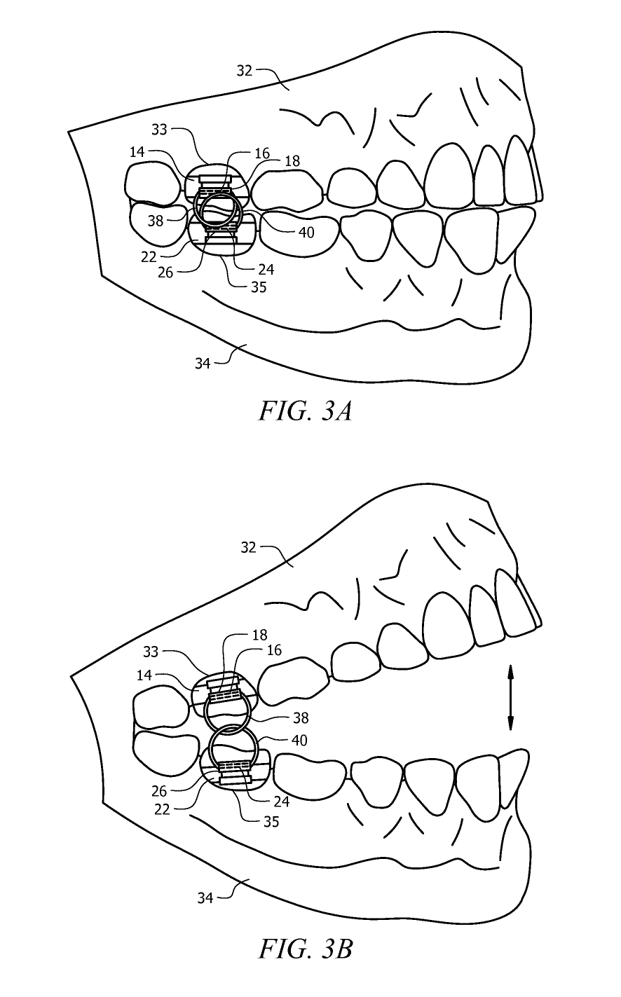

Referring to FIGS. 3A and 3B, an alternative embodiment of a jaw range limiter installed in a mouth will be described. An inside surface of a first tooth interface 14 is affixed to a first tooth 33 in the upper arch 32, and the inside surface of a second tooth interface 22 is affixed to a second tooth 35 in the lower arch 34. Each tooth interface 14, 22 is affixed in any way known in the industry to at least one tooth and/or faux tooth or dental support apparatus and/or structure, etc., as is known in the industry.

The tooth interfaces 14, 22 are affixed to opposing teeth 33, 35, although other installation locations are anticipated such as a neighboring tooth and/or faux tooth or dental support apparatus and/or structure, etc. as is known in the industry. A first bracket 18 is affixed to or integral to an outside surface of the first tooth interface 14 and a second bracket 26 is affixed to or integral to an outside surface of the second tooth interface 22.

A range limiting device is shown having a first D-shaped section 38 and a last D-shaped section 40. The first D-shaped section 38 of the range limiting device and the last D-shaped section 40 of the range limiting device are sequentially linked to each another. The first D-shaped section 20 of the range limiting device is movably affixed to a first enclosed channel 16 affixed to or integral to the first bracket 18. The last D-shaped section 40 of the range limiting device is movably affixed to a second enclosed channel 24 affixed to or integral to the second bracket 26.

In an alternative embodiment, prior to affixing the inside surface of each of the tooth interfaces 14, 22 to at least one tooth, each of the first D-shaped section 38 of the range limiting device and the last D-shaped section 40 of the range limiting device is sequentially linked to one another. Next, the first D-shaped section 38 of the range limiting device is movably affixed to the first enclosed channel 16, and the last D-shaped section 40 of the range limiting device is movably affixed to the second enclosed channel 24.

In an alternative embodiment not shown, a first C-shaped groove is affixed to or integral to the first tooth interface 14 and/or a second C-shaped groove is affixed to or integral to the second tooth interface 22. The first D-shaped section 38 of the range limiting device is movably affixed to the first C-shaped groove and the last D-shaped section 40 of the range limiting device is movably affixed to the second C-shaped groove affixed to or integral to the second tooth interface 22.

Preferably, the D-shaped sections 38, 40 of the range limiting device move freely while each is linked to one another and while each D-shaped section 38, 40 is movably affixed to an opposing enclosed channel 16, 24 and/or C-shaped groove (not shown). In FIG. 3A, the mouth is shown fully closed with the range limiting device installed. In FIG. 3B, the apparatus and system is shown physically limiting the mouth from opening to a range less than fully opened.

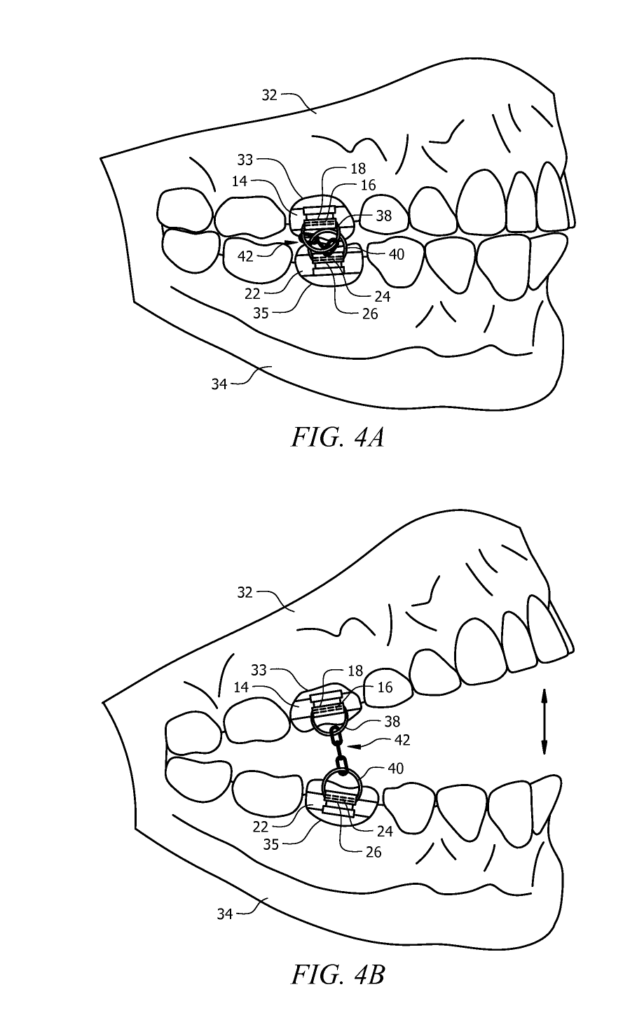

Referring to FIGS. 4A and 4B, an alternative embodiment of a jaw range limiter installed in a mouth will be described. An inside surface of a first tooth interface 14 is affixed to a first tooth 33 in the upper arch 32, and the inside surface of a second tooth interface 22 is affixed to a second tooth 35 in the lower arch 34. Each tooth interface 14, 22 is affixed in any way known in the industry to at least one tooth and/or faux tooth or dental support apparatus and/or structure, etc., as is known in the industry.

The tooth interfaces 14, 22 are affixed to opposing teeth 33, 35, although other installation locations are anticipated such as a neighboring tooth and/or faux tooth or dental support apparatus and/or structure, etc. as is known in the industry. A first bracket 18 is affixed to or integral to an outside surface of the first tooth interface 14 and a second bracket 26 is affixed to or integral to an outside surface of the second tooth interface 22.

A range limiting device is shown having three sections 38, 40, 42; a first semi-circular shaped section 38; a last semi-circular shaped section 40; and a third section 42 comprising three oval shaped sub-sections movably linked to one another. Each of the three sections 38, 40, 42 of the range limiting device may not be the same size and/or shape as shown in FIGS. 4A and 4B. Each of the three oval shaped sub-sections of the third section 42 of the range limiting device is shown smaller than both the first semi-circular shaped section 38 of the range limiting device and the last semi-circular shaped section 40 of the range limiting device. In an alternative embodiment, the third section 42 of the range limiting device comprises at least one oval shaped sub-section smaller than each of the first semi-circular shaped section 38 of the range limiting device and the last semi-circular shaped section 40 of the range limiting device. The first semi-circular shaped section 38 of the range limiting device is movably affixed to a first enclosed channel 16 affixed to or integral to the first bracket 18. The last semi-circular shaped section 40 of the range limiting device is movably affixed to a second enclosed channel 24 affixed to or integral to the second bracket 26.

In an alternative embodiment, prior to affixing the inside surface of each of the tooth interfaces 14, 22 to at least one tooth, each of the first semi-circular shaped section 38, the last semi-circular shaped section 40, and the third oval shaped sub-section is sequentially linked together. Next, the first semi-circular shaped section 38 is movably affixed to the first enclosed channel 16, and the last semi-circular shaped section 40 is movably affixed to the second enclosed channel 24. In an alternative embodiment not shown, a first C-shaped groove is affixed to or integral to the first tooth interface 14 and/or a second C-shaped groove is affixed to or integral to the second tooth interface 22. The first semi-circular shaped section 38 of the range limiting device is movably affixed to the first C-shaped groove and the last semi-circular shaped section 40 of the range limiting device is movably affixed to the second C-shaped groove affixed to or integral to the second tooth interface 22.

Preferably, each of the three sections 38, 40, 42 of the range limiting device move freely while linked to one another while both the first semi-circular shaped section 38 of the range limiting device is movably affixed to the first enclosed channel 16 and/or C-shaped groove (not shown) and the last semi-circular shaped section 40 of the range limiting device is movably affixed to the second enclosed channel 24 and/or C-shaped groove (not shown). In FIG. 4A, the mouth is shown fully closed with the range limiting device installed. In FIG. 4B, the apparatus and system is shown physically limiting the mouth from opening to a range less than fully opened.

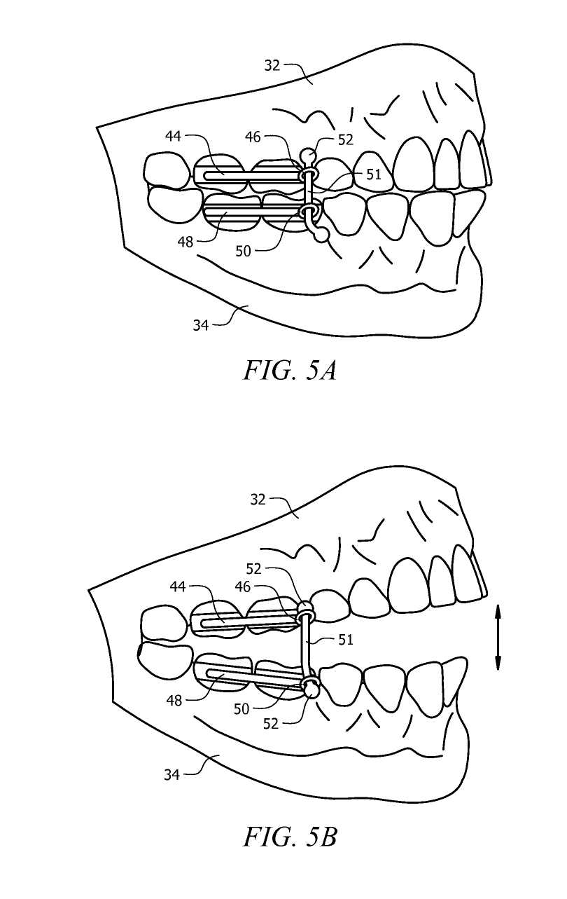

Referring to FIGS. 5A and 5B, an alternative embodiment of a jaw range limiter installed in a mouth will be described. An inside surface of a first tooth interface is affixed to a first tooth in the upper arch 32, and an inside surface of a second tooth interface is affixed to an adjacent second tooth in the upper arch 32. The neighboring tooth interfaces in the upper arch 32 are affixed to teeth, although faux teeth and/or other dental structures as are known in the industry are anticipated. A first horizontal bar 44 having a first loop 46 is affixed to or integral to both neighboring tooth interfaces in the upper arch 32. An inside surface of a third tooth interface is affixed to a third tooth in the lower arch 34, and an inside surface of a fourth tooth interface is affixed to a neighboring fourth tooth in the lower arch 34. A second horizontal bar 48 having a second loop 50 is affixed to or integral to both neighboring tooth interfaces in the lower arch 34.

A range limiting device shown comprises an elongated bar 51 having a protrusive first end 52 and a protrusive second end. Both the protrusive first end 52 of the elongated bar 51 and the protrusive second end of the elongated bar 51 are larger in diameter than the elongated bar 51. Also, although both the protrusive first end 52 and the protrusive second end are bulb and/or spherical shaped, other shapes are anticipated, such as cubical, triangular, etc.

First, the protrusive bulb on the first end 52 of the elongated bar 51 is removed and the elongated bar 51 is sequentially inserted through each of the first loop 46 affixed to or integral to the outside surface of the first tooth interface and the second loop 50 affixed to or integral to an outside surface of the second tooth interface. The protrusive bulb on the first end 52 of the elongated bar 51 is glued and/or affixed to the first end of the elongated bar 51 with a screwing and/or locking mechanism and/or any known way in the industry such that the protrusion is not easily removable. The protrusive bulb on the first end 52 of the elongated bar 51 is then re-affixed to the distal end of the elongated bar 51. The elongated bar 51 is slightly curved at the bottom, although other shapes and/or curvatures are anticipated, dependent upon the locations of opposing tooth interfaces and/or biomechanics of the opening and/or closing of the wearer's mouth. In FIG. 5A, the elongated bar 51 of the range limiting device is shown linking the first loop 46 and the second loop 50 in a closed mouth orientation.

Preferably, the range limiting device moves freely while the protrusive bulb on the first end 52 of the elongated bar 51 is movably affixed to the first loop 46 and the protrusive bulb on the second end of the elongated bar 51 is movably affixed to the second loop 50. In FIG. 5B, the apparatus and system is shown physically limiting the mouth from opening to a range less than fully opened.

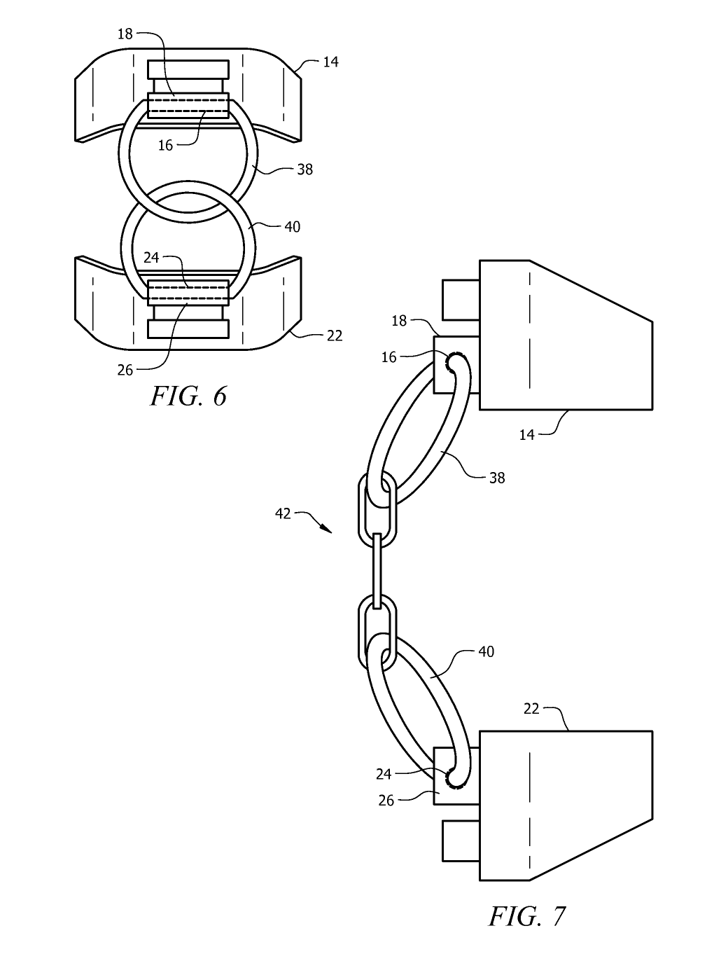

Referring to FIG. 6, an alternative embodiment of a jaw range limiter will be described. The jaw range limiter is shown having a first tooth interface 14, a first bracket 18 affixed to or integral to the first tooth interface 14, the first bracket 18 having a first enclosed channel 16 affixed to or integral to the first bracket 18, a second tooth interface 22, a second bracket 26 affixed to or integral to the second tooth interface 22, the second bracket 26 having a second enclosed channel 24 affixed to or integral to the second bracket 26. A range limiting device is shown having a first D-shaped section 38 of the range limiting device movably linked to a last D-shaped section 40 of the range limiting device.

Referring to FIG. 7, an alternative embodiment of a jaw range limiter will be described. The side view of the jaw range limiter is shown having a first tooth interface 14, a first bracket 18 affixed to or integral to the first tooth interface 14, a first enclosed channel 16 affixed to or integral to the first bracket 18, a second tooth interface 22, a second bracket 26 affixed to or integral to the second tooth interface 22, and a second enclosed channel 24 affixed to or integral to the second bracket 26.

A range limiting device is shown having a first semi-circular shaped section 38 of the range limiting device movably affixed to the first enclosed channel 16 and a last semi-circular shaped section 40 of the range limiting device movably affixed to the second enclosed channel 24. A third section of the range limiting device is shown comprising oval shaped sub-sections linking the first semi-circular shaped section 38 of the range limiting device and the last semi-circular shaped section 40 of the range limiting device. Each of the oval shaped sub-sections of the third section 42 of the range limiting device are smaller than each of the first semi-circular shaped section 38 of the range limiting device and the last semi-circular shaped section 40 of the range limiting device. In alternative embodiments, the third section 42 of the range limiting device comprises at least one oval shaped section smaller than each of the first semi-circular shaped section 38 of the range limiting device and the last semi-circular shaped section 40 of the range limiting device. Preferably, each of the first semi-circular shaped section 38 of the range limiting device, the last semi-circular shaped section 40 of the range limiting device, and the third sub-section 42 of the range limiting device move freely after each is sequentially linked to another of the sections and while each of the first semi-circular shaped section 38 of the range limiting device is movably affixed to the first enclosed channel 16 and the last semi-circular shaped section 40 of the range limiting device is movably affixed to the second enclosed channel 24.

In the foregoing embodiments, the range limiting device is made of materials known in the industry of sufficient strength and gauge, such that after the range limiting device is installed in the mouth of the wearer, the range limiting device is neither easily broken using normal jaw pressure, nor easily removable.

A jaw range limiter and method for weight loss has been shown which serves the purposes sought herein. Modifications, variations, other uses, and applications of the disclosure will become apparent to those skilled in the art after considering the specifications and the drawings. Modifications, variations, other uses, and applications not outside the scope and spirit of this disclosure are deemed covered by this disclosure.

Equivalent elements can be substituted for the ones set forth above such that they perform in substantially the same manner to achieve substantially the same results. It is believed that the apparatus and method of this disclosure and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and an explanatory embodiment thereof. It is the intention of the following claims to encompass and include such changes.

What is claimed is:

1. A dental apparatus for range limiting, the apparatus comprising:
a first tooth interface, an inside surface of the first tooth interface affixed to at least one upper tooth;
a second tooth interface, an inside surface of the second tooth interface affixed to at least one lower tooth;
a range limiting device having at least two sections, each of the at least two sections of the range limiting device is sequentially linked from a first section of the range limiting device to a last section of the range limiting device;
the first section of the at least two sections of the range limiting device is movably affixed to or integral to an outside surface of the first tooth interface;
the last section of the at least two sections of the range limiting device is movably affixed to or integral to an outside surface of the second tooth interface; and
whereas the first tooth interface and the second tooth interface are movably interconnected to one another, such that the range limiting device permits a wearer to completely close their mouth and the range limiting device limits the wearer to opening their mouth to a range less than fully opened.

2. A dental apparatus for range limiting of claim 1, further comprising a first bracket affixed to or integral to the outside surface of the first tooth interface, a first enclosed channel affixed to or integral to the first bracket, a second bracket affixed to or integral to the outside surface of the second tooth interface, a second enclosed channel, the second enclosed channel affixed to or integral to the second bracket, the first section of the at least two sections of the range limiting device is movably affixed to the first enclosed channel, and the last section of the at least two sections of the range limiting device is movably affixed to the second enclosed channel, thereby movably interconnecting the first tooth interface and the second tooth interface to one another, such that the range limiting device permits the wearer to completely close their mouth and the range limiting device limits the wearer to opening their mouth to a range less than fully opened.

3. A dental apparatus for range limiting of claim 1, further comprising a first loop affixed to or integral to the outside surface of the first tooth interface, and a second loop affixed to or integral to the outside surface of the second tooth interface, the first section of the at least two sections of the range limiting device is movably affixed to the first loop, and the last section of the at least two sections of the range limiting device is movably affixed to the second loop, thereby movably interconnecting the first tooth interface and the second tooth interface to one another, such that the range limiting device permits the wearer to completely close their mouth and the range limiting device limits the wearer to opening their mouth to a range less than fully opened.

4. A dental apparatus for range limiting of claim 1, further comprising a first C-shaped groove affixed to or integral to the outside surface of the first tooth interface, and a second C-shaped groove affixed to or integral to the outside surface of the second tooth interface, the first section of the at least two sections of the range limiting device is movably affixed to the first C-shaped groove, and the last section of the at least two sections of the range limiting device is movably affixed to the second C-shaped groove, thereby movably interconnecting the first tooth interface and the second tooth interface to one another, such that the range limiting device permits the wearer to completely close their mouth and the range limiting device limits the wearer to opening their mouth to a range less than fully opened.

5. A dental apparatus for range limiting of claim 1, wherein the at least two sections of the range limiting device comprise exactly two sections and each of the two sections of the range limiting device is selected from the group consisting of, a triangular shaped section, a semi-circular shaped section, a D-shaped section, a circular shaped section, or an oval shaped section, and wherein each of the two sections of the range limiting device are sequentially linked from the first section of the range limiting device to the last section of the range limiting device, thereby movably interconnecting the first tooth interface and the second tooth interface to one another, such that the range limiting device permits a wearer to completely close their mouth and the range limiting device limits the wearer to opening their mouth to a range less than fully opened.

6. A dental apparatus for range limiting of claim 1, wherein the at least two sections of the range limiting device comprise exactly three sections, wherein each of the first section of the range limiting device and the last section of the range limiting device is selected from the group consisting of, a triangular shaped section, a semi-circular shaped section, a D-shaped section, or a circular shaped section, and wherein a third section of the range limiting device is at least one other sub-section selected from the group consisting of, the circular shaped section, or the semi-circular shaped section, wherein each of the first section of the range limiting device, the last section of the range limiting device, and the third sub-section of the range limiting device are sequentially linked from the first section of the range limiting device to the last section of the range limiting device, such that the range limiting device permits complete closure of the mouth of the wearer and the range limiting device limits the opening of the mouth of the wearer to a range less than fully opened.

7. A dental apparatus for range limiting of claim 1, wherein the first tooth interface, the second tooth interface, and the range limiting device are made of materials known in the industry of sufficient strength and gauge, such that after the range limiting device is installed in a mouth of the wearer, the range limiting device is neither easily broken using normal jaw pressure, nor easily removable.

8. A method for weight loss, the method comprising:
providing a dental apparatus for range limiting, the dental apparatus for range limiting comprising:
affixing an inside surface of a first tooth interface to one upper tooth;
affixing an inside surface of a second tooth interface to one lower tooth;
providing a range limiting device having at least two sections;
sequentially linking each of the at least two sections of the range limiting device to another of the sections of the range limiting device from a first section of the range limiting device to a last section of the range limiting device;
before or after the step of sequentially linking the first section of the at least two sections of the range limiting to the last section of the at least two sections of the range limiting device, the step of movably affixing the first section of the range limiting device to an outside surface of the first tooth interface and the step of movably affixing the last section of the range limiting device to an outside surface of the second tooth interface;
whereas the range limiting device movably interconnects the first tooth interface and the second tooth interface to one another; and
whereas the range limiting device allows a wearer to completely close their mouth and the range limiting device limits the wearer to opening their mouth to a range less than fully opened.

9. The method of claim 8, further comprising a first enclosed channel affixed to or integral to the outside surface of the first tooth interface, a second enclosed channel affixed or integral to the outside surface of the second tooth interface, the first section of the at least two sections of the range limiting device is movably affixed to the first enclosed channel affixed to or integral to the outside surface of the first tooth interface, the last section of the at least two sections of the range limiting device is movably affixed to the second enclosed channel affixed to or integral to the outside surface of the second tooth interface, thereby movably interconnecting the first tooth interface and the second tooth interface to one another, such that the range limiting device allows a wearer to completely close their mouth and the range limiting device limits the wearer to opening their mouth to a range less than fully opened.

10. The method of claim 8, further comprising a first loop affixed to or integral to the outside surface of the first tooth interface, a second loop affixed or integral to the outside surface of the second tooth interface, the first section of the at least two sections of the range limiting device is movably affixed to the first loop affixed to or integral to the outside surface of the first tooth interface, the last section of the at least two sections of the range limiting device is movably affixed to the second loop affixed to or integral to the outside surface of the second tooth interface, thereby movably interconnecting the first tooth interface and the second tooth interface to one another, such that the range limiting device allows a wearer to completely close their mouth and the range limiting device limits the wearer to opening their mouth to a range less than fully opened.

11. The method of claim 8, further comprising a first C-shaped groove affixed to or integral to the outside surface of the first tooth interface, and a second C-shaped groove affixed or integral to the outside surface of the second tooth interface, the first section of the at least two sections of the range limiting device is movably affixed to the first C-shaped groove affixed to or integral to the outside surface of the first tooth interface, and the last section of the at least two sections of the range limiting device is movably affixed to the second C-shaped groove affixed to or integral to the outside surface of the second tooth interface, thereby movably interconnecting the first tooth interface and the second tooth interface to one another, such that the range limiting device allows a wearer to completely close their mouth and the range limiting device limits the wearer to opening their mouth to a range less than fully opened.

12. The method of claim 8, wherein the at least two sections of the range limiting device comprise exactly two sections and each of the two sections of the range limiting device is selected from the group consisting of, a triangular shaped section, a semi-circular shaped section, a D-shaped section, a circular shaped section, or an oval shaped section, and wherein each of the two sections of the range limiting device is sequentially linked from the first section of the range limiting device to the last section of the range limiting device, thereby movably interconnecting the first tooth interface and the second tooth interface to one another, such that the range limiting device allows a wearer to completely close their mouth and the range limiting device limits the wearer to opening their mouth to a range less than fully opened.

13. The method of claim 8, wherein the at least two sections of the range limiting device comprise exactly three sections, wherein each of the first section of the range limiting device and the last section of the range limiting device is selected from the group consisting of, a triangular shaped section, a semi-circular shaped section, a D-shaped section, or a circular shaped section, wherein a third section of the range limiting device is at least one other sub-section selected from the group consisting of, a circular shaped section, or a semi-circular shaped section, wherein each of the first section of the range limiting device, the last section of the range limiting device, and the third sub-section of the range limiting device is sequentially linked from the first section of the range limiting device to the last section of the range limiting device, thereby movably interconnecting the first tooth interface and the second tooth interface to one another, such that the range limiting device allows a wearer to completely close their mouth and the range limiting device limits the wearer to opening their mouth to a range less than fully opened.

14. The method of claim 8, wherein the first tooth interface, the second tooth interface, and the range limiting device are made of materials known in the industry having sufficient strength and gauge, such that after the range limiting device is installed in the wearer, the range limiting device is neither easily broken using normal jaw pressure, nor easily removable.

15. The method of claim 8, further comprising after the step of movably affixing the first section of the range limiting device to the outside surface of the first tooth interface, and the step of movably affixing the last section of the range limiting device to the outside surface of the second tooth interface, the step of periodic monitoring of the wearer and the step of periodic adjusting of the range limiting device, such that the range limiting device allows a wearer to completely close their mouth and the range limiting device limits the wearer to opening their mouth to a range less than fully opened.

16. A dental system for range limiting, the system comprising:
a first tooth interface, an inside surface of the first tooth interface affixed to at least one upper tooth;
a second tooth interface, an inside surface of the second tooth interface affixed to at least one lower tooth;
means for movably interconnecting the first tooth interface and the second tooth interface to one another; and
whereas the means for movably interconnecting the first tooth interface and the second tooth interface to one another is a range limiting device having at least two sections which are movably interconnecting the first tooth interface and the second tooth interface such that the system for range limiting allows a wearer to completely close their mouth and limits the wearer to opening of their mouth to a range less than fully opened.

17. A dental system as defined in claim 16, wherein each of the at least two sections is sequentially linked from a first section of the range limiting device to a last section of the range limiting device, the first section of the at least two sections of the range limiting device is movably affixed to an outside surface of the first tooth interface, the last section of the at least two sections of the range limiting device is movably affixed to an outside surface of the second tooth interface, thereby the means for movably interconnecting the first tooth interface and the second tooth interface to one another allows the wearer to completely close their mouth and limits the wearer to opening of their mouth to a range less than fully opened.

18. A dental system as defined in claim 16, wherein the means for movably interconnecting the first tooth interface and the second tooth interface to each other comprises a range limiting device, the range limiting device having an elongated bar, the elongated bar having a first protrusive bulb on a first end and a second protrusive bulb on a second end, at least one of either the first protrusive bulb or second protrusive bulb is removable from the elongated bar, each of the first protrusive bulb and second protrusive bulb is larger in diameter than the diameter of the elongated bar, whereby at least one of the protrusive bulbs is removed from the elongated bar and the elongated bar is then sequentially inserted through a first loop affixed to or integral to the an outside surface of the first tooth interface, and the elongated bar is inserted through a second loop affixed to or integral to an outside surface of the second tooth interface, the removed protrusive bulb is then re-affixed to the end of the elongated bar, thereby movably interconnecting the first tooth interface and the second tooth interface to one another, such that the system for range limiting allows the wearer to completely close their mouth and limits the wearer to opening of their mouth to a range less than fully opened.

19. A dental system as defined in claim 17, wherein each of the least two sections of the range limiting device comprises exactly two sections and each of the two sections of the range limiting device is selected from the group consisting of, a triangular shaped section, a semi-circular shaped section, a D-shaped section, a circular shaped section, or an oval shaped section.

20. A dental system as defined in claim 17, wherein each of an outside surface of the first tooth interface and an outside surface of the second tooth interface is selected from the group consisting of, an enclosed channel affixed to or integral to the outside surfaces of each of the first tooth interface and the second tooth interface, an enclosed channel affixed to or integral to a first bracket affixed to or integral to the outside surfaces of each of the first tooth interface and the second tooth interface, a loop affixed to or integral to the outside surfaces of each of the first tooth interface and the second tooth interface, or a C-shaped groove affixed to or integral to the outside surfaces of each of the first tooth interface and the second tooth interface, whereby the first section of the range limiting device is movably affixed to the outside surface of the first tooth interface and the last section of the range limiting device is movably affixed to the outside surface of the second tooth interface, thereby the system for range limiting allows the wearer to completely close their mouth and limits the wearer to opening their mouth to a range less than fully opened.
